SEQUENCE LISTING (3 sequences)

SEQ ID NO: 1 - DNA, 1379 bp, Artificial Sequence
  Nucleotide sequence of a DNA insert in pMTJA1
ggatccgaaattaatacgactcactatagggagaccacaacggtttccctctagaaataattttgtttaactttaagaaggagatatacatatgaaagctactaaactggtactgggcgcggtaatcctgggttctactctgctggcaggttgctccagcaacgctaaaatcgatcagggtatcgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatcccgggtggcgaaatccagaaaaaactggttgatccgagtaaatatggtaccaaatgtccgtataccatgaaaccgaaatatattaccgttcataacacctataatgatgcgccggcggaaaatgaagtgagctatatgattagcaacaataatgaagtgagctttcatattgcggtggatgataaaaaagcgattcagggtattccgctggaacgtaatgcgtgggcctgcggcgatggcaatggtagcggtaatcgtcagagcattagcgttgaaatttgctatagcaaaagcggcggtgatcgctactataaagccgaagataatgccgttgatgttgtgcgccagctgatgagcatgtacaatattccgattgaaaatgttcgtacccatcagagctggagcggtaaatattgcccgcatcgcatgctggccgaaggccgttggggtgcgtttattcagaaagttaaaaatggcaatgttgcgaccaccagcccgaccaaacagaacattatccagagcggtgcgtttagcccgtatgaaaccccggatgttatgggtgcgctgaccagcctgaaaatgaccgcggattttatcctgcagagcgatggcctgacctattttattagcaaaccgaccagcgatgcgcagctgaaagcgatgaaagaatatctggatcgtaaaggctggtggtatgaagttaaataagctt

SEQ ID NO: 2 - DNA, 698 bp, Artificial Sequence
  Sequence of gene coding for PlyG protein
gaaatccaaaaaaaattagttgatccaagtaagtatggtacaaagtgtccgtatacaatgaagcctaaatatatcactgttcacaacacatataatgatgctccagctgaaaatgaagtgagttacatgattagtaacaataatgaggtgtcgtttcatattgcagtagatgacaagaaagcgattcaaggtattccgttggaacgtaatgcatgggcttgcggagacggcaatggttcggggaatcgtcaatccatttctgtagaaatctgttattcaaaatcaggaggagatagatactataaagctgaggataatgctgttgatgttgtacgacaacttatgtctatgtacaatattcgattgaaaatgttcgaactcatcaatcctggtcaggtaaatattgtccgcatagaatgttagctgagggaaggtggggagcattcattcagaaggttaagaatgggaatgtggcgactacttcaccaacaaaacaaaacatcatccaatcaggggctttctcaccgtatgaaacccctgatgttatgggagcattaacgtcacttaaaatgacagctgattttatcttacaatcggatggattaacttattttatttccaaaccgacttcagatgcacaactaaaagcaatgaaagaataccttgaccgtaaaggttggtggtatgaagttaaataa

SEQ ID NO: 3 - DNA, 699 bp, Artificial Sequence
  Sequence to generate fusion gene in pMTJA1
gaaatccagaaaaaactggttgatccgagtaaatatggtaccaaatgtccgtataccatgaaaccgaaatatattaccgttcataacacctataatgatgcgccggcggaaaatgaagtgagctatatgattagcaacaataatgaagtgagctttcatattgcggtggatgataaaaaagcgattcagggtattccgctggaacgtaatgcgtgggcctgcggcgatggcaatggtagcggtaatcgtcagagcattagcgttgaaatttgctatagcaaaagcggcggtgatcgctactataaagccgaagataatgccgttgatgttgtgcgccagctgatgagcatgtacaatattccgattgaaaatgttcgtacccatcagagctggagcggtaaatattgcccgcatcgcatgctggccgaaggccgttggggtgcgtttattcagaaagttaaaaatggcaatgttgcgaccaccagcccgaccaaacagaacattatccagagcggtgcgtttagcccgtatgaaaccccggatgttatgggtgcgctgaccagcctgaaaatgaccgcggattttatcctgcagagcgatggcctgacctattttattagcaaaccgaccagcgatgcgcagctgaaagcgatgaaagaatatctggatcgtaaaggctggtggtatgaagttaaataa

(19) United States
(12) Patent Application Publication
(10) Pub. No.: US 2014/0308246 A1
(43) Pub. Date: Oct. 16, 2014

(54) Method Of Making Active Biological Containment Factors For Use In Selectively Killing Target Bacteria

(71) Applicant: CSU FULLERTON AUXILIARY SERVICES CORPORATION, Fullerton, CA (US)

(72) Inventors: Marcelo E. Tolmasky, Brea, CA (US); Juan C. Alonso Navarro, Colmenar Viejo (ES)

(73) Assignee: CSU Fullerton Auxiliary Services Corporation, Fullerton, CA (US)

(21) Appl. No.: 13/861,888

(22) Filed: Apr. 12, 2013

(51) Int. Cl. C12N 15/70

(52) U.S. Cl. CPC C12N 15/70
USPC 424/93.2; 435/6.18; 435/252.33

(57) ABSTRACT

The present invention provides a method of making biological containment factors for use in selectively killing target bacteria such as Bacillus anthracis, Clostridium botulinum, Clostridium perfringens, and other select agents.

### BACKGROUND OF THE INVENTION

1. Field of the Invention

The present invention is directed to a biological method of selectively killing target bacteria. More specifically, the present invention is directed to a method of making active biological containment factors designed specifically to kill target bacteria.

2. Background Art

Immediately following the tragic terrorist attacks of Sep. 11, 2001, an unknown person or group of people committed multiple acts of bioterrorism, Between September and November of 2001, the perpetrator(s) intentionally mailed weapons-grade, Bacillus anthracis spores to multiple sites within the United States. This act of terrorism resulted in five deaths, more than twenty infected individuals and panic throughout the US population. In addition to the death and disruption caused by these attacks, the United States and the international community received a wake up call on the need for increased emergency response readiness and safeguards designed to prevent or reduce the risk of casualties if another such attack occurs.

The anthrax spores delivered through the US postal service in 2001 resulted in over twenty contaminated sites. The four best decontamination methods available at that time included three chemical based treatments.

The chemical based methods rely on the use of toxic chemicals (e.g., Chlorine dioxide, Aklyl Dimethyl Benzyl Ammonium Chloride and Hydrogen Peroxide, ethylene oxide), which may be harmful to the personnel responsible for administering the process and to the structure or surfaces to be decontaminated.

In addition to the potential hazards created by the methods discussed above, the time involved in completing the decontamination process is potentially devastating. In the end, the cost of decontaminating all the facilities affected by the 2001 anthrax attack totaled over $1 billion.

Accordingly, there is a great need for a simple, cost effective and safe method for selectively killing deadly biological agents such as anthrax.

In recent years, researchers studying the characteristics of bacteriophage enzymes made a significant discovery. Contrary to the previous understanding that the lytic action of a bacteriophage would only occur from within a host cell as part of the bacteriophage's lifecycle, it was determined that certain bacteriophage lytic enzymes, such as PlyG, besides acting from within the cell host, they also mediate efficient lysis of the natural target bacterial host if from outside the bacteria's cell wall.

This concept has been exploited to develop a pharmaceutical composition comprising isolated and purified lytic enzyme polypeptides for specific prophylactic and therapeutic treatment of Bacillus anthracis. Although these isolated and purified lytic enzymes are effective for use in a pharmaceutical composition, the protein extraction and purification requirements, limit production volumes and rigid storage requirement limit its usefulness for any other application.

Accordingly, a need exists for a biological containment factor that provides the specificity of a lytic enzyme, which hydrolyzes the peptidoglycan of a cell wall, and the ease of production and storage found in the current chemical methods.

### SUMMARY OF THE INVENTION

The present invention provides a safe, stable, cost effective and selective biological containment factor for use in killing specific target bacteria without harming the surrounding biological material or underlying structure.

More specifically, the present invention provides a biological containment factor comprising a biological agent transformed to express a derivative of an enzyme known to promote lysis of the cell wall of the identified target bacterium.

The present invention further provides a method of making a biological containment factor comprising modifying a biological agent to express a derivative of an enzyme which is known to promote lysis of a selected target bacterium.

In addition, the present invention provides a method of biological containment comprising contacting the selected target bacteria with the biological containment factor to initiate lysis of the selected target bacteria.

Furthermore, the present invention provides a method of biological containment comprising applying the biological containment factor to a surface contaminated with selected target bacteria to initiate lysis of the selected target bacteria wherein the biological containment factor will be selectively killed after exerting its action.

These and other aspects and objects of the present invention will be better appreciated and understood when considered in conjunction with the following figures and description. It should be understood, however, that the following description while indicating the preferred embodiments of the present invention and numerous specific details thereof, is given by way of illustration and not of limitation. Many changes and modifications may be made within the scope of the present invention without departing from the spirit thereof, and the invention includes all such modifications.

### BRIEF DESCRIPTION OF THE DRAWINGS

The various embodiments, features and advances of the present invention will be understood more completely hereinafter as a result of a detailed description thereof in which reference will be made to the following drawings:

FIG. 1 depicts a flow chart of the method of making and use of the biological containment factor;

FIG. 2A depicts a physical and genetic map of cloning vehicle pUC57;

FIG. 2B depicts a physical and genetic map of cloning vehicle pFUS2;

FIG. 2C depicts a physical and genetic map of recombinant clone pMTJA1 which includes cloning vehicle pUC57 modified to contain a DNA insert harboring a hybrid gene “biological containment agent” and a fusion protein;

FIG. 2D depicts a physical and genetic map of recombinant clone pMTJA2, which includes cloning vehicle pFUS2 modified to contain a DNA insert harboring a hybrid gene “biological containment agent” and a fusion protein;

FIG. 2E depicts a physical and genetic map of recombinant clone pMTJA3, which includes cloning vehicle pUC57 modified to contain a DNA insert harboring a hybrid gene “biological containment agent” and the ζ toxin and ε antitoxin genes (viable but non-culturable (VENC) cassette);

FIG. 2F depicts a physical and genetic map of recombinant clone pMTJA4, which includes cloning vehicle pFUS2 modified to contain a DNA insert harboring a hybrid gene “biological containment agent”, and the ζ toxin and antitoxin genes (viable but non-culturable (VBNC) cassette);

FIG. 3A depicts a schematic representation of a DNA insert in the recombinant clone pMTJA1;

FIG. 3B depicts Sequence No. 1, a nucleotide sequence of DNA (hybrid gene) insert in pMTJA1;

FIG. 4 depicts nucleotide sequence of the gene coding for the PlyG protein (accession number AF536823), Sequence No. 2, compared to a corresponding sequence constructed to generate the fusion gene in pMTJA1, Sequence No. 3; and

FIG. 5 depicts growth inhibition of Bacillus cereus BGCS 6A5.I

### BRIEF DESCRIPTION OF A PREFERRED EMBODIMENT

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

The present invention is directed to a method of making and use of biological containment factors to selectively kill target bacteria. More specifically, the biological containment factor comprises a biological agent transformed to express a recombinant protein that includes in its structure a lytic enzyme known to lyse a corresponding target bacterium. Once a biological containment factor expressing the desired recombinant protein is identified, it is propagated with conventional techniques.

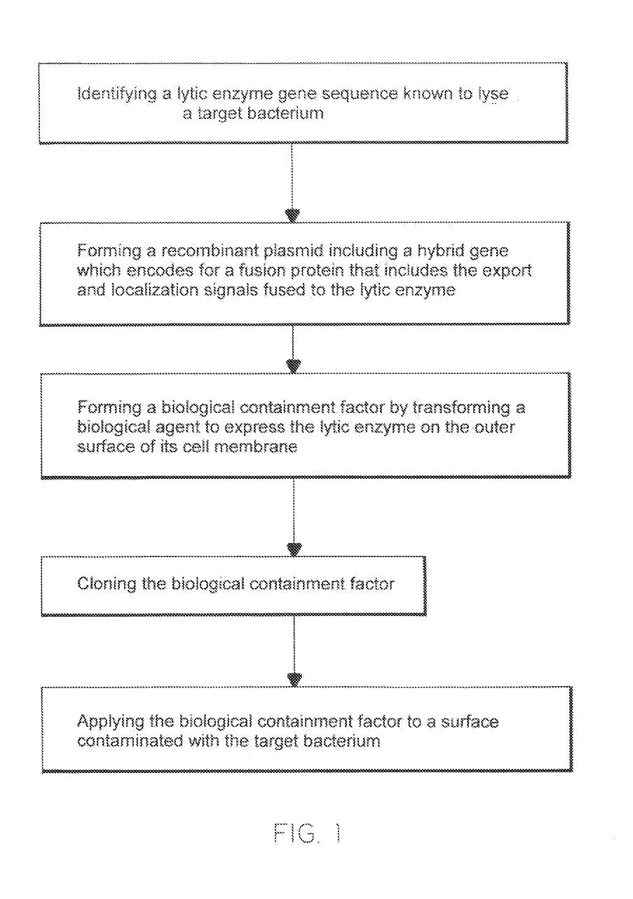

Referring now to FIG. 1, the method of making and use of the biological containment factor of the present invention includes the steps of identifying a lytic enzyme gene sequence known to lyse a target bacterium, forming a recombinant plasmid including a hybrid gene designed to express a fusion protein including the export and localization signals fused to the lytic enzyme. Forming a biological containment factor by transforming a biological agent to express the fusion lytic enzyme on the outer surface of the cell. Selecting and propagating the biological containment factor, and applying the biological containment factor to a surface contaminated with the target bacterium.

Once a target bacterium to be killed is selected, a lytic enzyme known to effectively lyse the target bacterium must be identified. Bacteriophage lytic enzymes, also referred to as endolysins, are bacteriophage-encoded enzymes that hydrolyze the peptidoglycan of a cell wall to release mature phage particles formed within the host cell. These lytic enzymes also mediate efficient lysis of the target bacterium from the outside. If a target bacterium and specific bacteriophages are well cataloged, the appropriate lytic enzyme gene sequence will be easily identified and obtained for use in a hybrid gene sequence. See Table 1 for a correlation of examples of potential target bacteria and described bacteriophages.

| Select agents^{a} | Bacteriophage families^{b} |
|---|---|
| Bacillus anthracis | Siphoviridae |
| Brucella abortus | Podoviridae morphotype C1 |
| Brucella melitensis | Podoviridae morphotype C1 |
| Brucella suis | Podoviridae morphotype C1 |
| Burkholderia mallei (formerly | Myoviridae morphotype A1, A2 |
| Pseudomonas mallei) | |
| Burkholderia pseudomallei | Myoviridae morphotype A1, A2 |
| (formerly Pseudomonas | |
| pseudomallei) | |
| Clostridium perfringens | Siphoviridae morphotype B1, Inoviridae, |
| epsilon toxin | Myoviridae morphotype A1, A2 |
| Clostridium botulinum | Siphoviridae morphotype B1, Inoviridae, |
| neurotoxins | Myoviridae morphotype A1, A2 |
| Coxiella burnetii | Myoviridae morphotype A1, A2 |
| Francisella tularensis | Myoviridae morphotypes A1, A2 |
| Rickettsia prowazekii | Podoviridae morphotype C1 |
| Rickettsia prowazekii | Podoviridae morphotype C1 |
| Rickettsia rickettsii | Podoviridae morphotype C1 |
| Staphylococcus aureus | Siphoviridae morphotype B1, B2 |
| enterotoxins | |
| Yersinia pestis | Myoviridae morphotypes A1, A2 |

^{a}http://www.cdc.gov/od/sap/docs/salist.pdf
^{b}http://www.mansfield.ohio-state.edu/~sabedon/names.htm, http://www.phage.org http://www.ebi.ac.uk/genomes/phage.html.

In the case of endolysin-resistant bacteria a specific bacteriophage known to infect the selected bacterium can be used. The lytic enzyme is then produced and analyzed to ensure that it will be active when supplied from the outside of the target bacterium.

If no bacteriophage is known for use with the described bacterium, a bacteriophage can be isolated from natural sources.

In the case that the genome has not yet been sequenced, the lytic enzyme will be identified by immobilizing the substrate in a polyacrylamide gel matrix followed by separation of the proteins from a crude extract obtained from the infected bacteria using sodium dodecyl sulfate polyacrylamide gel electrophoresis, protein renaturation in the gel, followed by in situ determination of enzyme activity. The spot showing activity contains the lytic enzyme. The N-terminus will then be determined and the corresponding nucleotide will be deduced to design a probe for gene amplification and recombinant cloning of the lytic enzyme gene.

To determine the preferred enzyme for a given bacterium, one needs to identify the level of knowledge about an identified bacteriophage. If the bacteriophage's genome is known, the lytic enzyme's amino acid sequence can be deduced using appropriate software.

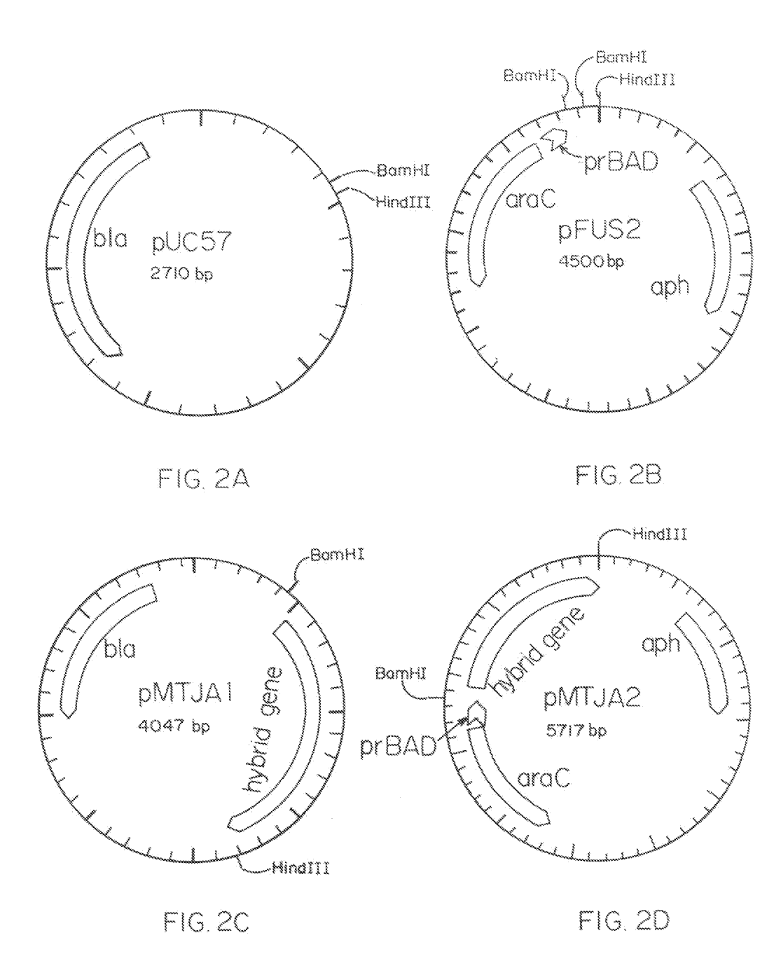

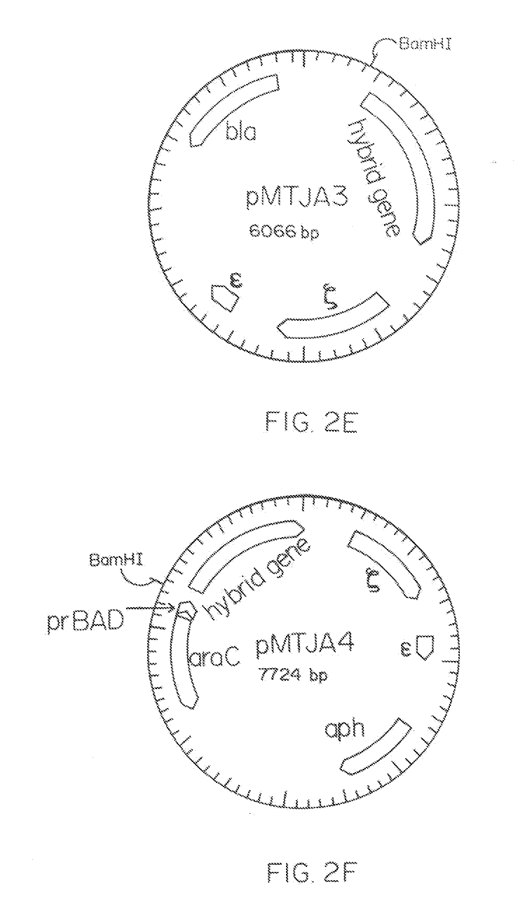

After a lytic enzyme known to lyse a target bacterium is identified, a recombinant plasmid containing a hybrid of the lytic enzyme gene sequence is formed. More specifically, an isolated DNA fragment known to encode for the selected lytic enzyme is joined to another coding for localization signals to form a hybrid gene which encodes for a fusion protein that includes the lytic activity and it is exposed to the cell's surface. A cloning vector, such as pUC57 as shown in FIG. 2A or pFUS2 as shown in FIG. 2B, is then modified with the hybrid gene to form a recombinant plasmid such as pMTJA1 as shown in FIG. 2C or pMTJA2 as shown in FIG. 2D respectively. A host biological agent, such as E. coli, is then transformed with the recombinant plasmid.

The cloning vector may be selected from any vector capable of being modified to form a recombinant plasmid, but is preferably pUC57 or pFUS2.

The biological agent is any non-pathogenic bacterium that can be modified to produce and express a fusion protein-gene hybrid on its surface. Although any non-pathogenic bacterium such as E. coli, other proteobacteria, and firmicutes may be used, E. coli is preferred.

The resulting transformed biological agents or “biological containment factors” are then screened to identify those that produce the desired lytic enzyme on the outer surface of the cell. The biological containment factors found to produce the lytic enzyme on the outer surface of the cell are then selected, propagated, and ready for use in the decontamination process.

Although the cloning vectors and biological agents used in the present invention are environmentally safe, (e.g., vectors lacking transfer functions and biological agents that are mutants having an obligate requirement for exogenous nutrients), the level of safety of the biological containment factor can be increased by reducing the possibility of biological containment factor's survival. To achieve this, the cloning vector such as pUC57 as shown in FIG. 2A or pFUS2 as shown in FIG. 2B, is modified with both the hybrid gene and a gene coding for a cell function that induces the“viable but non-culturable” (VBNC) state to produce a recombinant plasmid containing the hybrid gene and the VBNC cassette as shown in FIGS. 2E and 2F, respectively. More specifically, the VBNC cassette includes the stable ζ cytotoxin (287 amino acids long) polypeptide with “phosphotransferase” activity that interacts with a labile small ε antitoxin (90 amino acids long) polypeptide that antagonizes the toxic effect of the ζ cytotoxin. A biological agent is then transformed with the resulting recombinant plasmid as described above.

In use, this VBNC cassette is under the control of a promoter that is only expressed under certain environmental conditions. Although bacterial cells grow normally under conditions in which the VBNC cassette is expressed, a selective repression of the expression of the antitotoxin (for example, removal of isopropyl-thio-β-D-galactopyranoside [IPTG] from the media) will induce the VBNC state on the large majority of the cells.

The biological containment factor formed through the process described above comprises a biological agent transformed with a recombinant plasmid to express a selected lytic enzyme on the outer surface of the cell. Since the biological containment factor is active, it can be easily propagated or cloned and either used immediately or stored for later use.

When the biological containment factor is used for decontamination, it is applied to a surface contaminated with the target bacterium either alone or in the presence of a composition formulated to induce the growth of the targeted bacteria or bacteria producing spores. For example, a growth enhancing composition such as Tryptic Soy Broth supplemented with outgrowth inducers may be used to enhance spore outgrowth. The lytic enzyme expressed on the external surface of the biological containment factor then comes into contact with the target bacteria, and lyses its cell wall thereby killing it.

### EXAMPLE 1

Bacillus anthracis is selected as the target bacterium. Accordingly, bacteriophage γ, which is known to lyse Bacillus anthracis is selected to provide the lytic enzyme, PlyG.

PlyG, the endolysin used by the bacteriophage γ to mediate host cell lysis at the end of the lytic cycle, also mediates cell lysis when added to B. anthracisor to the closely related Bacillus cereus strain BGSC 6A5, PIyG is a ˜27 kDa murein hydrolase that presents two domains. The domain located at the N-terminal portion is catalytic and possesses N-acetylmuramoyl-L-alanine amidase activity. The other domain is located at the C-terminus and binds the cell wall in a specific manner.

Of the biological agents available for modification, E. coli is selected because is the most versatile microorganism and is the best developed tool or molecular techniques. Other biological agents that can be modified to produce and express a hybrid gene coding for a protein fusion that includes the export and localization signals fused to the lytic enzyme include E. coli, other proteobacteria, or firmicutes. If the lytic enzyme is properly expressed, the transformed E. coli strain should have the ability to inhibit growth or kill B. anthracis or similar Bacillus strains susceptible to infection with the bacteriophage γ, such as Bacillus cereus BGCS 6A5.

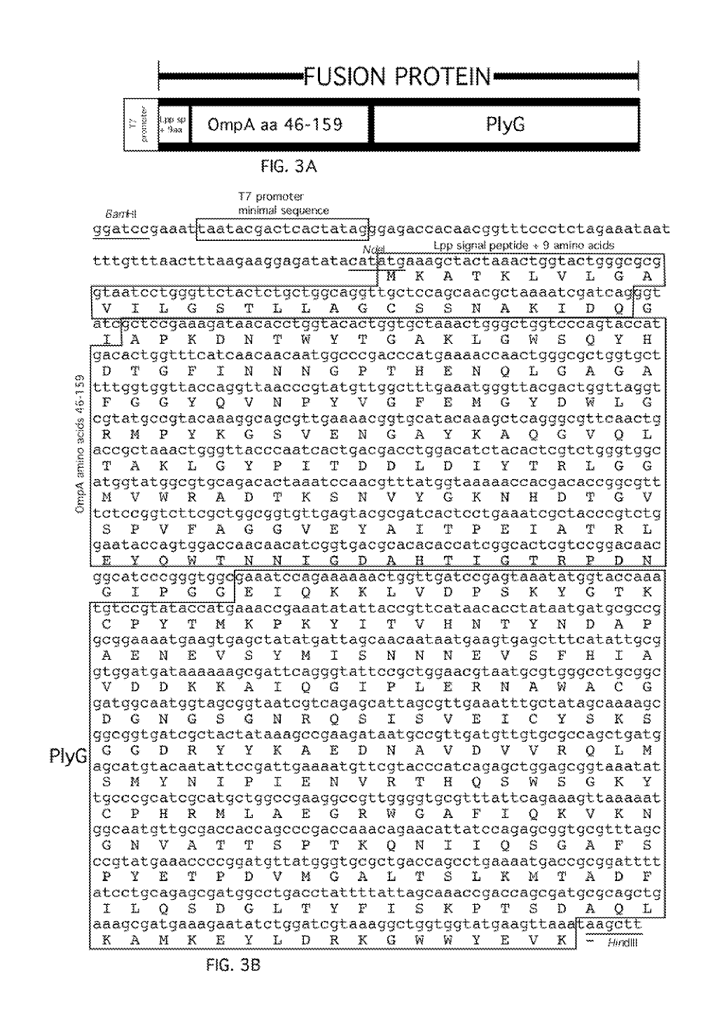

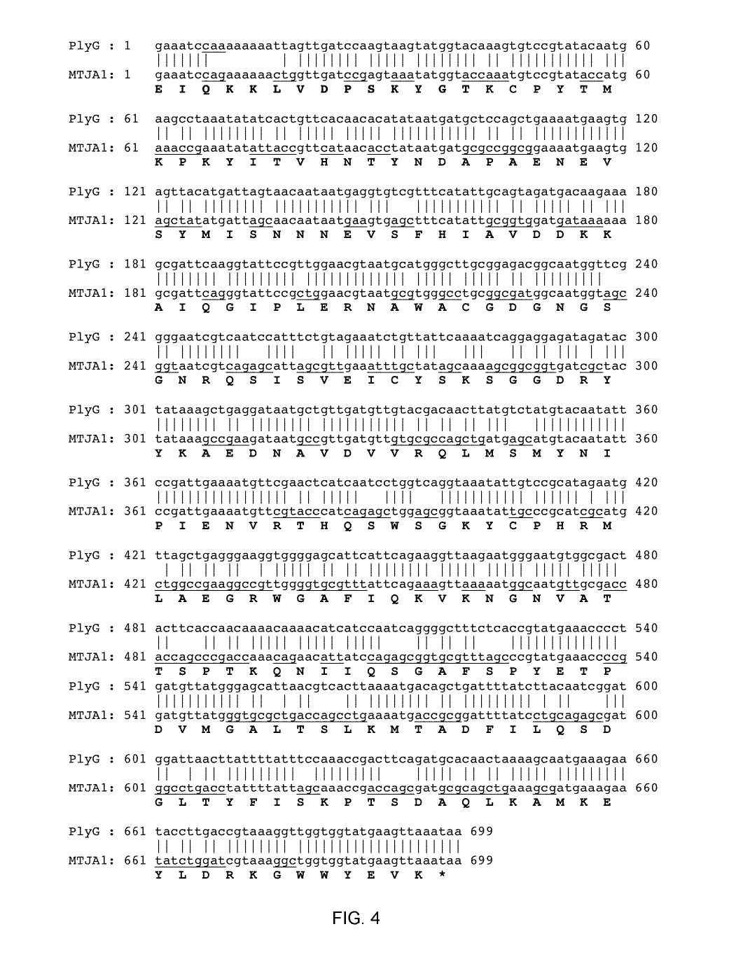

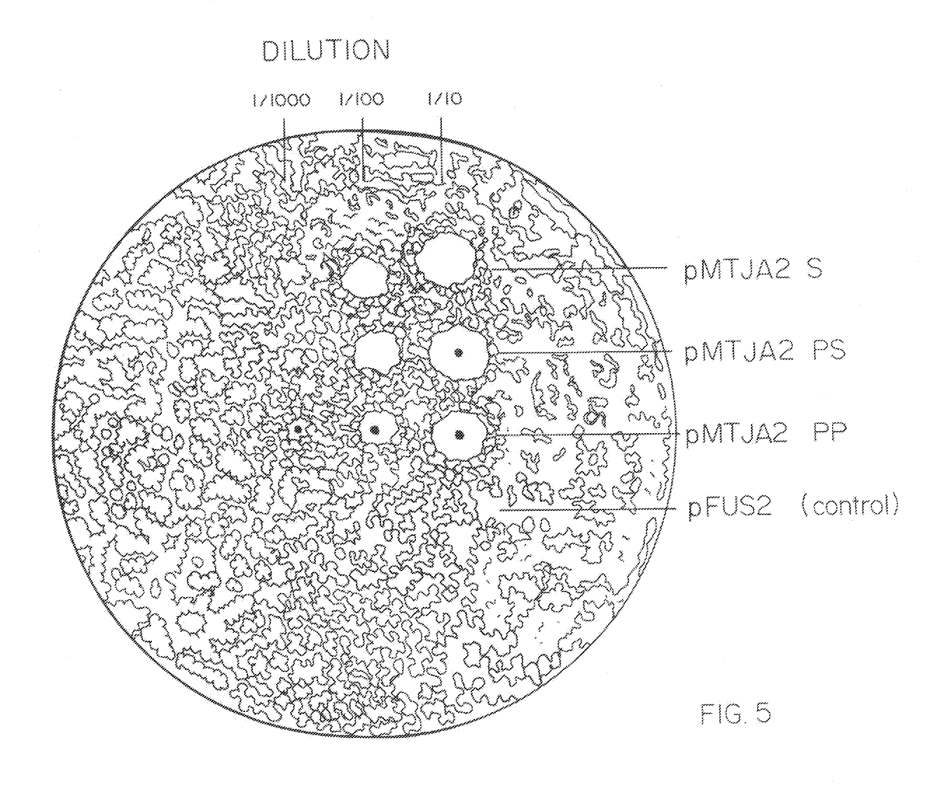

To construct the E. coli strain capable of expressing PlyG the cloning vector pUC57 as shown in FIG. 2A, is utilized to generate the recombinant plasmid pMTJA1 as shown in FIG. 2C. This recombinant plasmid contains a DNA insert harboring an hybrid gene designed to code for a fusion protein composed of the docker (a signal peptide plus the first 9 amino acids of the mature Lpp protein, a 2-amino acids spacer, the membrane-spanning region of OmpA encompassing amino acids 46-159, a 5-amino acids spacer), and the endolysin moiety (the complete amino acid sequence of PIyG) as shown in FIGS. 3A and 3B. To ensure proper expression of the PlyG in E. coli BL21(DE3) cells (or any strain containing the gene 1 of the T7 bacteriophage), the composition of several codons were modified according to the E. coli codon usage, without modifying the amino acid sequence of the protein as shown in FIG. 4.

In addition, the plasmid is further modified by addition of the VBNC cassette to make the cells addict to an antitoxin in such a way that in its absence the plasmid bearing cells will die as a consequence of the cytotoxic effect. This will induce the death of the cells once they have fulfilled their decontamination function. To make the cells addict to an inducer, such as IPTG, the toxic gene is placed under the control of a constitutive promoter (P_{ω}), taken from plasmid pCB298, and the antitoxin gene, taken from pCB297, is placed under the control of a regulatory DNA sequence (for example the lac promoter, P_{lacO}). Therefore, in the absence of the appropriate inducer (for example IPTG), expression of the gene coding for the antitoxin polypeptide is suppressed leading to death of the cells. The and genes modified as described will be inserted into pMTJA1 as shown in FIG. 2C, to generate pMTJA3 as shown in FIG. 2E.

Alternatively, the hybrid gene is cloned under the control of the BAD promoter (P_{BAD}) instead of a T7 promoter. For this purpose the pMTJA1 was cleaved with BamHI and HindIII and the 1,379 by DNA fragment encompassing the ORF shown in FIG. 3B was joined to BamHI-HindIII-cleaved pFUS2 expression vector (FIG. 2B), as shown in FIG. 2D, to generate plasmid pMTJA2. In pMTJA2 the ORE is under the control of P_{BAD} and its expression is under the control of arabinose. Plasmid pMTJA2, is introduced into E. coli strain CC118 and cultured in the presence of kanamycin (50 μg/ml). In this case the plasmid pMTJA2 will also be modified by insertion of the and genes as described above, to generate pMTJA4 as shown in FIG. 2F.

The resulting biological containment factor was then tested as follows to determine if the strains can interfere with B. cereus growth:

### Experiment 1

Plasmid pMTJA1 DNA is introduced into E. coli XL1-Blue and BL21(DE3) competent cells and cultured in the presence of ampicillin (75 μg/ml). Colonies harboring plasmids with the appropriate size (˜24% of total transformants) are selected for further analysis. The nucleotide sequences of two of the selected recombinant clones are confirmed by dideoxynucleotide sequencing. E. coli XL1-Blue (pMTJA1 or pMTJA3) and E. coli BL21(DE3)(pMTJA1) are stored and maintained at −80° C. E coli BL21(DE3) harboring pMTJA1 or the control plasmid pT712, a pUC-like plasmid bearing a T7 promoter, are grown in LB to OD₅₆₀ 0.6 at 37° C. with shaking, then the cultures are divided into two aliquots. One aliquot of each of the cultures is then induced by the addition of 0.5 mM IPTG followed by incubation in the same conditions for 30 min at 37° C.

After incubation each culture is split in two aliquots and the cells are harvested by centrifugation at 5,000 rpm at 4° C. In one of the aliquots the supernatant (called 5) is separated form the pellet containing the cells (called PS). The pellet is then resuspended in 1/10, 1/100 and 1/1000 of the original volume in buffer A (50 mM tris-HCl pH 7.5, 500 mM NaCl, 2 mM EDTA, 5% glycerol) or buffer B (50 mM tris-HCl pH 7.5, 40 mM NaCl, 10 mM MgCl₂, 5% glycerol). In the other aliquot, after centrifugation the cells are lysed by sonication and centrifuged again at 5,000 rpm at 4° C. The resulting pellet is washed twice with the resuspension buffer and resuspended in 1/10, 1/100 and 1/1000 of original volume on buffer A or buffer B. This fraction bearing mainly insoluble protein and cell debris is called PP. All three fractions; original supernatant (5), original pellet bearing whole cells (PS) and pellet of the lysed cells (PP) are used to test their ability to interfere with growth of B. cereus in seeded plates as follows.

B. cereus is cultured to OD₅₆₀ 0.1 in L-broth (LB) at 37° C. with shaking. A 100 μl aliquot of the culture is then plated on a 100 mm diameter L-agar plate. Then, 10 μl aliquots of each extract as described in the previous paragraph are spotted over the B. cereus lawn and the plates are incubated overnight at 37° C. Lysis halos are detected with the highest concentration ( 1/10) of the extracts (see Table 1). However, it was observed that after more than 36 hours incubation, the B. cereus strain could overgrow the halo suggesting that the expression of Docker-PlyG (FIG. 3B) from pMTJA1 was poor Experiments using higher extract concentration (> 1/10) could not be performed because the controls induced B. cereus lysis.

As shown in Table 2 below, B. cereus lysis is detected in the S and PS fractions up to a dilution of 1/100 using both buffer compositions, buffer B being more efficient (compare dilutions 1/100 in Table 2). However, when independent assays were done using several sibling colonies, in about 40% of them we failed to detect B. cereus lysis. Upon further analysis it was observed that in 80% of the cases in which no lysis was observed, the recombinant plasmid contained a DNA insert smaller than the sequence originally inserted indicating that the negative results were correlated with the absence of the Docker-PIyG fusion hybrid gene probably due to spontaneous deletions or rearrangements.

TABLE 2

| Con- | (S) | (PS) | (PP) | (S) | (PS) | (PP) |
|---|---|---|---|---|---|---|
| dition | pMTJA1 | pMTJA1 | pMTJA1 | pT712 | pT712 | pT712 |
| buffer | ++ | ++ | − | − | − | − |
| A | | | | | | |
| 1/100 | | | | | | |
| buffer | ++ | ++ | − | − | − | − |
| A | | | | | | |
| 1/10 | | | | | | |
| buffer B | ++ | ++ | − | − | − | − |
| 1/100 | | | | | | |
| buffer B | +++ | +++ | − | − | − | − |
| 1/10 | | | | | | |

+++, full lysis,
++, partial lysis,
+, poor lysis and,
−, no detectable lysis.

When the E. coli cells bearing pMTJA3-borne gene ζ under the control of a constitutive promoter and ε gene under the control of an IPTG-dependent promoter were plated in an IPTG-free media, the reduction in plating efficiency decrease 10⁶- to 10⁷-fold.

### Experiment 2

E. coli CC118 competent cells are transformed with pMTJA2 (FIG. 2D) plasmid DNA and selected in the presence of kanamycin (50 μg/ml). About 87% of the transformant colonies harbored recombinant plasmid with the appropriate size. The plasmid present within one of the transformant colonies is subjected to nucleotide sequencing to confirm that the insert is present. These cells are stored at −80° C. in 50% glycerol. The E. coli CC118 cells harboring pMTJA2 or plasmid vector pFUS2 are cultured in LB to OD₅₆₀ 0.6 at 37° C. with shaking. The cultures are then divided into two aliquots. One aliquot of each of the cultures is induced by the addition of 1% arabinose followed by incubation in the same conditions for 30 min at 37° C.

The arabinose containing culture is then divided into two aliquots and the resulting cells are harvested by centrifugation at 5,000 rpm at 4° C. In one of the aliquots the supernatant (S) is separated to from the pellet (PS). The pellet was resuspended in 1/10, 1/100 and 1/1000 of original volume in buffer B. In the other aliquot the cells are lysed by sonication and centrifuged at 5,000 rpm at 4° C. The resulting pellet is washed twice with buffer B and resuspended in 1/10, 1/100 and 1/1000 of original volume on buffer B. This fraction is called PP. All three fractions; original supernatant (S), original pellet (PS) and pellet of the lysed cells (PP) are used to test their ability to interfere with growth of B. cereus in seeded plates. Both cells extracts are then resuspended.

B. cereus is cultured to OD₅₆₀ 0.1 in L-broth (LB) at 37° C. with shaking. A 100 μl aliquot of the culture is then plated on a 100 mm diameter L-agar plate. Then, 10 μl aliquots of each E. coli CC118 (pMTJA2) extract obtained as described in the previous paragraph are spotted over the B. cereus lawn and the plates were incubated overnight at 37° C. Lysis halos are detected even in the minimal concentration of the extracts from cells harboring pMTJA2, but not with pFUS2 (FIG. 5). This suggests that there is enough expression of Docker-PlyG “hybrid gene” in E. coli CC118 (pMTJA2). In addition the stability of the pMTJA2 recombinant plasmid when compared with pMTJA1 seems to be higher.

The results show the biological agent modified through the process of the present invention displays effective killing activity through intact modified cells as well as with the culture supernatant and whole cell membrane.

When the E. coli cells bearing pMTJA4-borne ζ gene under the control of a constitutive promoter and ε gene under the control of an IPTG-dependent promoter were plated in an IPTG-free media, the reduction in plating efficiency decrease 10⁶- to10⁷-fold.

### EXAMPLE 2

If the target bacteria are Clostridium perfringens and Clostridium botulinum, a gene coding for a protein similar to that described in the previous section is engineered. More specifically, the biological containment factor would expose the phage φ 3626-encoded peptidoglycan hydrolase to the outer surface of the cell. The 5′ end of the C. perfringens phage φ 3626-encoded peptidoglycan hydrolase (endolysin, ORF 20) gene, whose product cleaves peptidoglycan in a species-specific manner, is fused to the Docker. The resulting biological containment factor expresses the fused protein composed of the docker and the phage φ 3626-encoded peptidoglycan hydrolase, able to selectively kill C. perfringens and C. botulinum cells.

While this invention has been described with specific embodiments thereof, it is evident that many alternatives, modifications, and variations will be apparent to those skilled in the art. Accordingly, the preferred embodiments of the invention as set forth herein are intended to be illustrative, not limiting. Various changes may be made without departing from the spirit and scope of the invention.

We claim:

1. A method of making a biological containment factor comprising:
identifying a lytic enzyme gene sequence known to lyse a target bacterium;
forming a recombinant plasmid including a hybrid gene coding for the membrane localization signals and the lytic enzyme and a fusion protein (biological containment agent); and
forming the biological containment factor by transforming the biological containment agent to express the lytic enzyme on the outer surface of the cell.

2. The method of claim 1, wherein the recombinant plasmid includes a hybrid gene designed to express the membrane localization signals and the lytic enzyme as a fusion protein (biological containment agent) and a VBNC cassette.

3. The method of claim 1, wherein the lytic enzyme gene sequence is PlyG.

4. The method of claim 1, wherein the biological agent expresses phage φ 3626-encoded peptidoglycan hydrolase on the outer surface of its cell membrane.

5. The method of claim 1, wherein the recombinant plasmid is pMTJA1.

6. The method of claim 1, wherein the recombinant plasmid is pMTJA2.

7. The method of claim 1, wherein the recombinant plasmid is pMTJA3.

8. The method of claim 1, wherein the recombinant plasmid is pMTJA4.

9. A biological containment factor for selectively killing a target bacterium comprising:
a biological containment agent transformed to express a lytic enzyme on the outer surface of the cell, wherein the lytic enzyme is known to lyse the target bacterium.

10. The biological containment factor of claim 9, wherein the target bacterium is Bacillus anthracis or Bacillus cereus.

11. The biological containment factor of claim 9, wherein the target bacterium is Clostridium perfringens or Clostridium botulinum.

12. A method of selectively killing a target bacterium comprising:
contacting the target bacterium with a biological containment factor formed to express a lytic enzyme known to lyse the target bacterium.

13. The method of claim 12, further comprising inducing growth of the target bacterium.

14. The method of claim 13, wherein the biological containment factor formation comprises:
identifying a lytic enzyme gene sequence known to lyse a target bacterium;
forming a recombinant plasmid including a hybrid gene encoded to express the lytic enzyme and a fusion protein; and
forming the biological containment factor by transforming a biological agent to express the lytic enzyme on the outer surface of the cell.

15. The method of claim 14, wherein the recombinant plasmid includes a hybrid designed to express the membrane localization signals and the lytic enzyme as a fusion protein (biological containment agent) and a VBNC cassette.

16. The method of claim 14, wherein the recombinant plasmid is pMTJA1.

17. The method of claim 14, wherein the recombinant plasmid is pMTJA2.

18. The method of claim 14, wherein the recombinant plasmid is pMTJA3.

19. The method of claim 14, wherein the recombinant plasmid is pMTJA4.
